(19) United States
(12) Patent Application Publication
(10) Pub. No.: US 2014/0336476 A1
(43) Pub. Date: Nov. 13, 2014

(54) MICROCHIP SENSOR FOR CONTINUOUS MONITORING OF REGIONAL BLOOD FLOW

(71) Applicant: THE FEINSTEIN INSTITUTE FOR MEDICAL RESEARCH, Manhasset, NY (US)

(72) Inventor: Chunyan Li, Franklin Square, NY (US)

(73) Assignee: THE FEINSTEIN INSTITUTE FOR MEDICAL RESEARCH, Manhasset, NY (US)

(21) Appl. No.: 14/354,712

(22) PCT Filed: Oct. 26, 2012
(86) PCT No.: PCT/US12/62096

§ 371 Date: Apr. 28, 2014

Related U.S. Application Data
(60) Provisional application No. 61/552,855, filed on Oct. 28, 2011.

(51) Int. Cl. A61B 5/0205; A61B 5/00

(52) U.S. Cl. CPC A61B 5/02055; A61B 5/6852; A61B 5/021
USPC 600/301; 600/381

(57) ABSTRACT

A sensor is provided available for continuous monitoring of regional blood flow in a tissue, including cerebral tissue. Methods of monitoring regional blood flow using the sensor as well as systems and computer readable medium therefor are also provided.

### CROSS-REFERENCE TO RELATED APPLICATIONS

This application claims benefit of U.S. Provisional Application No. 61/552,855, filed Oct. 28, 2011, the contents of which are hereby incorporated by reference.

### STATEMENT OF GOVERNMENT SUPPORT

This invention was made with government support under grant number W81XWH-1-09 awarded by the U.S. Department of Defense. The government has certain rights in the invention.

### BACKGROUND OF THE INVENTION

Throughout this application various patents and other publications are referred to in parenthesis. Full citations for the references may be found at the end of the specification. The disclosures of these patents and publications are hereby incorporated by reference in their entirety into the subject application to more fully describe the art to which the subject invention pertains.

The common mechanism for brain injury-induced neuronal loss is inadequate cerebral blood flow (CBF) for the neurons. During pathological conditions such as traumatic brain injury (TBI) or subarachnoid hemorrhage (SAH), CBF is considered an important upstream monitoring parameter that is indicative of tissue viability (1-3). Hence, monitoring of CBF plays an important role in neurosurgical practice. Continuous monitoring of CBF could provide the opportunity to diagnose and to correct insufficient CBF before deficits in tissue oxygenation and metabolism are recognized. Many techniques are available to assess CBF, such as stable xenon-enhanced computed tomography, single-photon-emission computed tomography, magnetic resonance imaging, positron emission tomography and laser-doppler flometry. However, few of these techniques lend themselves to routine clinical application due to enduring technical drawbacks. Recently, the thermal diffusion flowmetry-based measurement technique which allows the direct and quantitative assessment of regional cerebral perfusion represents a promising monitoring tool in the management of head injured patients (4-5).

The present invention address the need for improved devices and methods to assess and/or monitor regional blood flow in a tissue, especially cerebral blood flow.

### SUMMARY OF THE INVENTION

The invention provides a sensor for monitoring blood flow in a biological tissue, the sensor comprising (a) a flow sensor comprising a first microelectrode, which flow sensor is capable of being heated by conduction of electric current through the microelectrode, and (b) a temperature sensor comprising a second microelectrode, wherein (a) and (b) are arranged on a flexible substrate and wherein (a) is positioned relative to (b) such that when (a) is heated to a stable target temperature of 0.5° C. to 3° C. above the temperature of the tissue in which the sensor is situated, (b) is not within the temperature field generated by (a).

In another aspect of the invention, methods are provided for determining a blood flow in a tissue of subject comprising measuring the blood flow using the sensor for monitoring blood flow, as described herein, situated in the tissue of the subject.

A system is also provided for monitoring a blood flow in a subject comprising: one or more data processing apparatus coupled to a sensor for monitoring blood flow as described herein; and a computer-readable medium coupled to the one or more data processing apparatus having instructions stored thereon which, when executed by the one or more data processing apparatus, cause the one or more data processing apparatus to perform a method as described herein so as to monitor the blood flow in a subject.

In another aspect of the invention, a non-transitory computer-readable medium is provided comprising instructions stored thereon which, when executed by a data processing apparatus, causes the data processing apparatus to perform a method as described herein so as to monitor blood flow in a subject.

Additional objects of the invention will be apparent from the description which follows.

### BRIEF DESCRIPTION OF THE DRAWINGS

FIG. 1A-1B. (A) Schematics of the smart catheter flow sensor (SCF). (B) Working principle: periodic heating technique is used to do in situ temperature and thermal conductivity compensation.

FIG. 2A-2B. Photograph: (A) microfabricated smart catheter (ID: 1.3 mm; OD: 1.65 mm) with temperature and flow sensors and (B) developed flow sensor signal conditioning interface circuit.

FIG. 3A-3B. Thermodynamic equilibrium tests: (A) timing diagram: cooling period (I, II) for temperature measurement is 6 seconds and heating period for thermal conductivity and flow measurements (III, IV, V) is 4 seconds; and (B) SCF outputs at the different cooling and heating periods.

FIG. 4. Temperature compensation during the heating period: The middle line indicates the medium temperature change (drop rate) measured by the integrated SCT. The uncompensated outputs are higher than the compensated outputs. In contrast, the compensated output remains constant regardless of the medium temperature change.

FIG. 5A-5C. Thermal conductivity compensation tests: (A) timing diagram in the medium with different thermal conductivity (glucose solution: 0.621, 0.571, 0.534 and 0.461 W·m-1·K-1); (B) calibration curve of thermal conductivity versus applied squared current; and (C) Applied squared current at different flow rates.

FIG. 6A-6B. Relationship between the flow sensor outputs and the flow rates: (A) continuous recording waveform at different flow rates and (B) calibration curve. The sensitivity is 0.973 mV/ml/100 gram-min in the range from 0 to 180 ml/100 gram-min.

FIG. 7. Long-term stability test: the outputs of SCF were recorded at the flow rate of 30 ml/100 gram-min for 5 days.

FIG. 8. Temperature compensation tests: Hot water is poured and mixed to induce immediate 3° C., 5° C. and 8° C. temperature changes. With the temperature compensation scheme described, the SCF outputs are returned to their original level. However, without temperature compensation, the SCF outputs are never returned to their original levels.

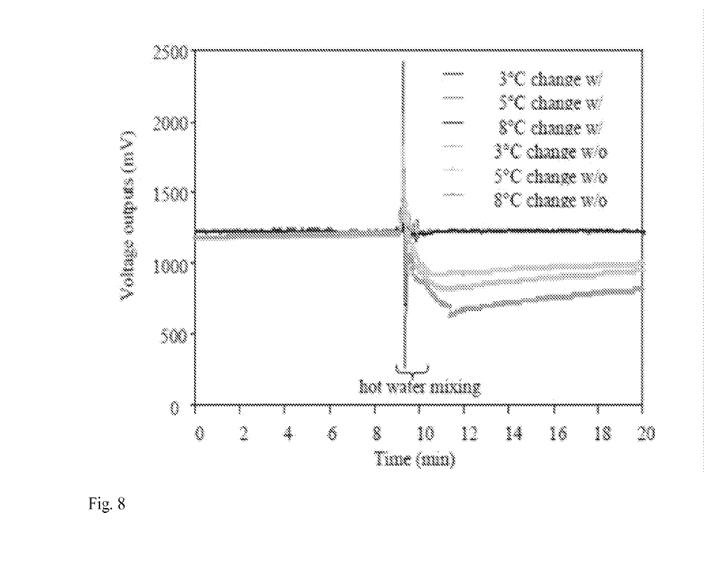

FIG. 9. Thermal conductivity measurement: A constant current was applied to increase the SCF resistance to the point of 2° C. above the medium temperature. The rate at which the resistance increased was affected by the medium thermal conductivity.

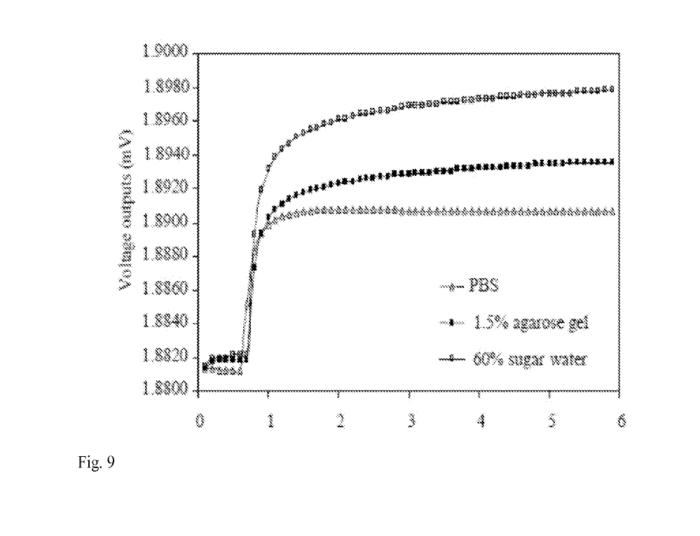

### DETAILED DESCRIPTION OF THE INVENTION

Abbreviations:
SCF—smart catheter flow sensor
CBF—cerebral blood flow
SCT—separate temperature sensor

As used herein a “sensor” is a device that measures a physical quantity and converts it into a signal which can be read by an observer or by an instrument. In the case of a temperature sensor, temperature is being measured. In the case of a liquid flow sensor, flow is calculated from another parameter measured, such as temperature. The device may be a microelectrode-based device, for example a thermoresistive microelectrode-based device. The output is an electrical output (e.g. a signal) which is related, for example proportional, to the parameter being determined, such as temperature.

As used herein a “biological tissue” is any tissue with a blood supply in an animal, or which has been removed from an animal As such, biological tissue includes, in non-limiting examples, a tissue in situ and a tissue which has been removed from, e.g. a human, for transplant purposes.

As used herein a “flexible substrate” is any suitable substrate which does not evoke an inflammatory and/or immune response when in situ in most subjects. While it is not possible to exclude any one individual from having an inflammatory and/or immune response to a foreign material, many materials are accepted to be generally inert. Such materials which are flexible and can be used as flexible substrates are known in the art. Examples of one class of flexible substrates that can be used in the present invention are polyimide, poly(p-xylylene), and polyvinylidene fluoride trifluoroethylene (PDVF-TrFE), poly-lactic-co-glycolic acid (PLGA), polyethylene, and polydimethylsiloxane (PDMS).

The invention provides a sensor for monitoring blood flow in a biological tissue, the sensor comprising (a) a flow sensor comprising a first microelectrode, which flow sensor is capable of being heated by conduction of electric current through the microelectrode, and (b) a temperature sensor comprising a second microelectrode, wherein (a) and (b) are arranged on a flexible substrate and wherein (a) is positioned relative to (b) such that when (a) is heated to a stable target temperature of 0.5° C. to 3° C. above the temperature of the tissue in which the sensor is situated, (b) is not within the temperature field generated by (a).

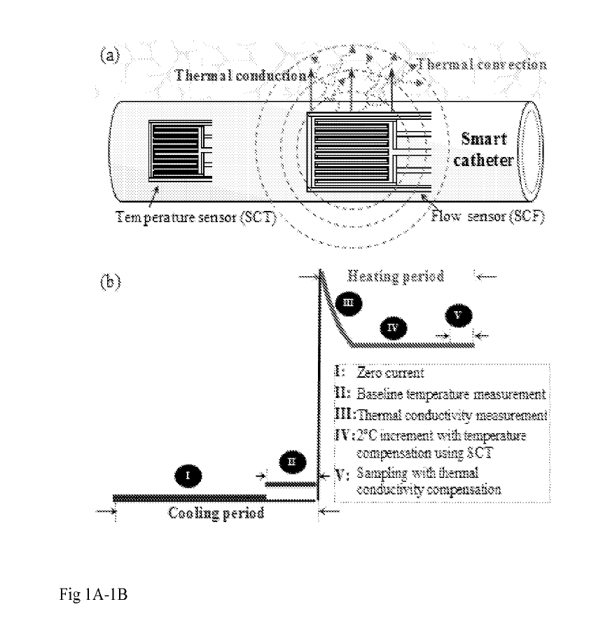

The temperature field generated by (a), represented schematically in FIG. 1(a), is the area around (a) within which an actual and measurable increase in temperature occurs upon (a) being heated to a predetermined stable target temperature.

The sensor for monitoring blood flow is small enough in size to be inserted into blood vessels. In an embodiment the sensor for monitoring blood flow is 10 μm-25 μm thick. In a preferred embodiment the device comprising the sensor for insertion into a tissue is no more than 20 μm thick. In a most preferred embodiment the device comprising the sensor for insertion into a tissue is no more than 15 μm thick. In an embodiment the device comprising the sensor for insertion into a tissue is 10 μm-25 μm thick.

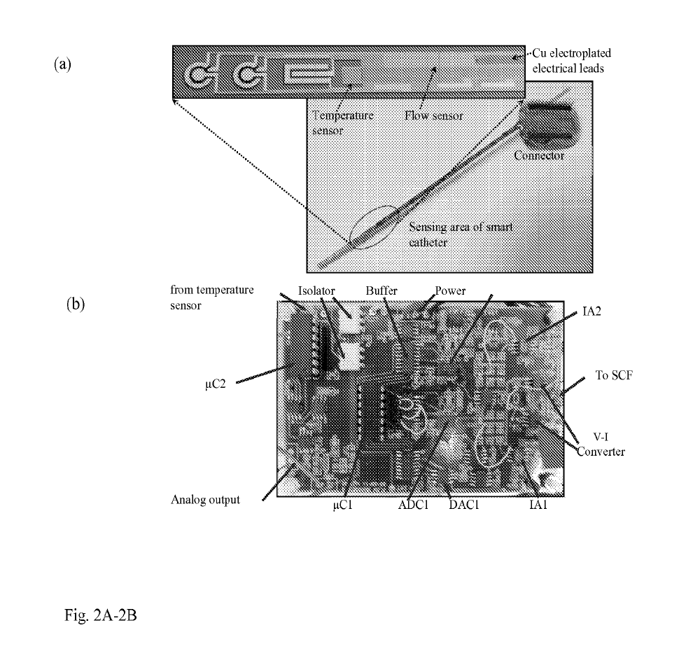

The flexibility of the sensor reduces tissue damage when the sensor is placed and aids insertion into blood vessels. The sensor can be fabricated using any means known in the art, including, in a non-limiting example, spirally rolling technology. For example, the sensor can be fabricated as a flexible spirally-rolled polymer microtube, the fabrication of which is described in U.S. Patent Application Publication No. 2009/0297574 A1, the contents of which are hereby incorporated by reference. In a preferred embodiment, the sensor for monitoring blood flow is fabricated as a catheter for blood vessels, or is fabricated to be placed inside a catheter for blood vessels. FIG. 2A shows a non-limiting exemplary fabrication.

The sensor for monitoring blood flow can be part of or integrated into an assembly further comprising one or more of a pressure sensor, a pH sensor, a glucose sensor, a microdialysis probe, an oxygen sensor, a lactate sensor, a pyruvate sensor, a glutamate sensor, and/or a carbon dioxide sensor.

In a preferred embodiment, the sensor for monitoring blood flow is operated to ratiometrically measure the resistance of each the sensors of which it is comprised. This results in a more precise measurement than bridge-type thermal diffusion flow sensors. In a preferred embodiment the flow sensor comprising a first microelectrode comprises a 4-wire configuration. This advantageously eliminates lead wire effect. In a preferred embodiment the temperature sensor comprising a first microelectrode comprises a 4-wire configuration

In an embodiment, the temperature sensor quantitates the temperature of the medium in which sensor for monitoring blood flow is situated, and wherein the sensor for monitoring blood flow is a component of an electrical circuit such that the output of the flow sensor is corrected for changes in temperature of the medium by the output of the temperature sensor. In a most preferred embodiment, the medium comprises the biological tissue. Accordingly, in an embodiment, the sensor for monitoring blood flow is a constant-temperature flow sensor, which compensates for medium baseline temperature shifts and thus provides improved accuracy of measurement. In a preferred embodiment, the electrical circuit comprises an interface circuit.

The microelectrodes of the sensor for monitoring blood flow are each continuous with electrically-conducting wires which are preferably electroplated with a material to reduce lead resistances thereof. Suitable electroplating material for the wires includes copper. In an embodiment, the wires are coated with copper 1 to 4 μm thick. In a preferred embodiment, the wires are coated with copper 1.5 to 2.5 μm thick. In a most preferred embodiment, the wires are coated with copper 2 μm thick. The gold can be can be fabricated by depositing one or more conducting materials, such as metals, on a suitable base such as an insulator. In a preferred embodiment the microelectrodes comprise gold. In a preferred embodiment the microelectrodes further comprise titanium. In an embodiment the gold is deposited in a layer 800-1600 Å thick, for example, on a flexible insulator. Suitable insulators include, in a non limiting example, a poly(4,4′-oxydiphenylene-pyromellitimide). In a preferred embodiment, the gold is deposited in a layer 1000-1400 Å thick. In a most preferred embodiment, the gold is deposited in a layer 1200 Å thick. In a preferred embodiment, the titanium is deposited on top of the gold in a layer 80-200 Å thick. In a preferred embodiment, the titanium is deposited in a layer 120-180 Å thick. In a most preferred embodiment, the titanium is deposited in a layer 150 Å thick. In an embodiment, the insulator is 5-15 μm thick. In a preferred embodiment, the insulator is 6-10 μm thick. In a most preferred embodiment, the insulator is 7.5 μm thick. The microelectrodes may be completed using thin film lithography and etching processes known in the art. The components may further be coated with one or more poly(p-xylylene) polymers. In an embodiment, the poly(p-xylylene) polymer is 2-10 μm thick. In a preferred embodiment, the poly(p-xylylene) polymer is 4-7 μm thick. In a most preferred embodiment, the poly(p-xylylene) polymer is 5 μm thick.

In the sensor for monitoring blood flow, (a) is capable of being heated to a steady temperature above the temperature of the medium. In an embodiment, it is capable of being heated by electrical current passed through it to a temperature of 0.1° C. to 3.5° C. above the temperature of the tissue in which the sensor is situated for a continuous time period of up to 20 seconds. In a preferred embodiment, it is capable of being heated by electrical current passed through it to a temperature of 1° C. to 2.5° C. above the temperature of the tissue in which the sensor is situated for a continuous time period of up to 20 seconds. In a most preferred embodiment, it is capable of being heated by electrical current passed through it to a temperature of 2° C. above the temperature of the tissue in which the sensor is situated for a continuous time period of up to 20 seconds.

In a preferred embodiment, the sensor for monitoring blood flow is operationally connected to a control device which controls the input current and/or voltage into the temperature sensor and which controls the input current and/or voltage into the flow sensor. In a most preferred embodiment, the sensor is operationally connected to one or more data processors which receive and process the output of the flow sensor and/or temperature sensor. The data processors can, and preferably do, compensate the output of the flow sensor for changes in the temperature of the medium (e.g. changes in the local temperature of the brain) as determined from the output of the temperature sensor.

In an embodiment, the sensor for monitoring blood flow can also correct for the thermal conductivity of the medium in which the device is placed. In a preferred embodiment, the one or more data processors further compensates the output of the flow sensor for changes in the thermal conductivity of the medium as determined from the peak output of the flow sensor during a period in which (a) is heated.

In an embodiment, the thermal conductivity of the medium is determined from sampling the peak initial current required to heat (a) to a stable target temperature of 1° C. to 3° C. above the temperature of the tissue in which the sensor is situated. In a preferred embodiment, the thermal conductivity of the medium is determined from sampling the peak initial current required to heat (a) to a stable target temperature of 2° C. above the temperature of the tissue in which the sensor is situated.

In an embodiment, the peak initial current is sampled when the flow sensor temperature is 0.05° C. to 0.3° C. above the stable target temperature. In a preferred embodiment, the peak initial current is sampled when the flow sensor temperature is 0.1° C. to 0.2° C. above the stable target temperature. The peak initial current can be sampled for any appropriate time period. In an embodiment, the peak initial current is sampled for 25 mS to 200 mS. In a preferred embodiment, the peak initial current is sampled for 75 mS to 125 mS. In a most preferred embodiment, the peak initial current is sampled for 100 mS. To reduce error, the thermal conductivity of the medium is preferably determined from the square of average of the sampling of two peak initial currents.

The blood flow rate is preferably determined from the output of the flow sensor compensated for thermal conductivity of the medium and compensated for changes in temperature of the medium. Such a method will give the most accurate determination. The invention as described shows a very good correlation voltage output is proportional to the blood flow rate. In preferred embodiments, the linear coefficient of R² for the correlation is in excess of 0.95. In a most preferred embodiment, the linear coefficient of R² for the correlation is in excess of 0.99.

The blood flow can be measured using the described sensor in any tissue. In a preferred embodiment, the tissue is a cerebral tissue and the blood flow is a cerebral blood flow.

In another aspect of the invention methods are provided for determining a blood flow in a tissue of subject comprising measuring the blood flow using the sensor for monitoring blood flow, as described herein, situated in the tissue of the subject.

In an embodiment, a baseline tissue temperature is determined comprising causing a non-heating current to be applied to the sensor for monitoring blood flow and measuring the output of the sensor. In an embodiment, the non-heating current is applied for between 0.1 and 20 seconds. In a preferred embodiment, the non-heating current is applied for between 2 and 8 seconds. In a more preferred embodiment, the non-heating current is applied for between 3 and 6 seconds.

In embodiments, the method also comprises effecting a temperature of the flow sensor to stably be 0.5° C. to 3° C. above the temperature of the tissue, for example cerebral tissue, in which the sensor is situated by causing a heating current to be applied to the sensor. In a preferred embodiment, a stable temperature of 1° C. to 2.5° C. above the temperature of the tissue is effected. In a most preferred embodiment, a stable temperature of 2° C. above the temperature of the tissue is effected. In an embodiment, to effect a temperature of the flow sensor to be above the temperature of the tissue, the heating current is applied for between 0.1 and 20 seconds. In a preferred embodiment, the heating current is applied for between 2 and 8 seconds. In a most preferred embodiment, the heating current is applied for 3 seconds, 4 seconds or for a time period in between 3 and 4 seconds.

The output of the flow sensor is preferably measured during the posterior portion of the period during which the heating current is applied. In an embodiment, measuring the output of the flow sensor is effected during the last 0.1 to 8 seconds of the period during which the heating current is applied. In an embodiment, measuring the output of the flow sensor is effected during the last 0.1 to 6 seconds of the period during which the heating current is applied. In an embodiment, measuring the output of the flow sensor is effected during the last 0.1 to 5 seconds of the period during which the heating current is applied. In an embodiment, measuring the output of the flow sensor is effected during the last 0.1 to 4 seconds of the period during which the heating current is applied. In a preferred embodiment, measuring the output of the flow sensor is effected during the last 0.1 to 3 seconds of the period during which the heating current is applied. In a most preferred embodiment, measuring the output of the flow sensor is effected during the last 0.5 to 2 seconds of the period during which the heating current is applied. In an embodiment, measuring the output of the flow sensor is effected during the last 1 second of the period during which the heating current is applied. In an embodiment, the output of the flow sensor during the last third, last quarter or last fifth of the period during which the heating current is applied.

In embodiments, the device and/or methods described herein are used in the early detection of vasospasm and other conditions of compromised perfusion. In an embodiment of the methods, the tissue is cerebral tissue. In a preferred embodiment, the device and/or methods described herein are used in monitoring cerebral blood flow when managing secondary injury in traumatic brain injury subjects. In another preferred embodiment, the device and/or methods described herein are used in monitoring cerebral blood flow during neurosurgical applications, for example during neurosurgery upon the subject and/or in the recovery period after neurosurgery upon the subject. In a preferred embodiment, the subject is a human.

The reading can be corrected for by compensating for the thermal conductivity of the tissue. In an embodiment, the method comprises determining the thermal conductivity of the cerebral tissue by sampling the peak initial current required to heat the flow sensor to stably be 1° C. to 3° C. above the temperature of the cerebral tissue. In a preferred embodiment, the method comprises determining the thermal conductivity of the cerebral tissue by sampling the peak initial current required to heat the flow sensor to stably be 1.5° C. to 2.5° C. above the temperature of the cerebral tissue. In a most preferred embodiment, the method comprises determining the thermal conductivity of the cerebral tissue by sampling the peak initial current required to heat the flow sensor to stably be 2° C. above the temperature of the cerebral tissue.

The method can be used to continuously or discontinuously monitor blood flow as desired. In an embodiment, the tissue blood flow measured at a plurality of discrete time points using the sensor for monitoring blood flow.

Preferably, the sensor for monitoring blood flow is permitted to cool after termination of a first heating current and initiation of a second or subsequent heating current being applied. In an embodiment, the period during which the sensor for monitoring blood flow is permitted to cool is 1 to 20 seconds. In a preferred embodiment, the period during which the sensor for monitoring blood flow is permitted to cool is 2 to 10 seconds. In a most preferred embodiment, the period during which the sensor for monitoring blood flow is permitted to cool is 2 to 5 seconds.

A baseline cerebral tissue temperature can be determined by causing a non-heating current to be applied to the sensor for monitoring blood flow and measuring the output of the sensor during the period during which the sensor for monitoring blood flow is permitted to cool. This permits monitoring of the baseline temperature so as to re-calibrate the sensor for any changes in baseline temperature. In an embodiment, the method comprises determining baseline cerebral tissue temperature during the terminal portion of the time period during which the sensor for monitoring blood flow is permitted to cool and immediately before the a heating current is applied.

In an embodiment of the sensor, the sensor is constructed such that the sensor is capable of being heated to a stable target temperature of 1° C. to 3° C. above the temperature of the tissue and subsequently cooling to the temperature of the tissue within a total period of 5-10 seconds. In an embodiment, the sensor is constructed such that the sensor is capable of being heated to a stable target temperature of 1.9° C. to 2.1° C. above the temperature of the tissue and subsequently cooling to the temperature of the tissue within a total period of 5-10 seconds.

In an embodiment of the method the sensor is heated to a stable target temperature of 1° C. to 3° C. above the temperature of the tissue and subsequently cooled to the temperature of the tissue within a total period of 5-10 seconds. In an embodiment of the method the sensor is heated to a stable target temperature of 1.9° C. to 2.1° C. above the temperature of the tissue and subsequently cooled to the temperature of the tissue within a total period of 5-10 seconds.

A system is also provided for monitoring a blood flow in a subject, comprising:

one or more data processing apparatus coupled to a sensor for monitoring blood flow as described herein; and

a computer-readable medium coupled to the one or more data processing apparatus having instructions stored thereon which, when executed by the one or more data processing apparatus, cause the one or more data processing apparatus to perform a method as described herein so as to monitor the blood flow in a subject. In a preferred embodiment, the blood flow is a cerebral blood flow. In an embodiment, the computer-readable medium is non-transitory.

In another aspect of the invention, a non-transitory computer-readable medium is provided comprising instructions stored thereon which, when executed by a data processing apparatus, causes the data processing apparatus to perform a method as described herein so as to monitor blood flow in a subject. In a preferred embodiment, the blood flow is a cerebral blood flow.

In another aspect of the devices, sensors, methods, and systems described herein, the blood flow is determined in a subject has suffered a brain injury or is undergoing a surgery or a therapeutic intervention upon the brain.

In embodiments, the device and/or methods described herein are used to monitor blood flow in a non-cerebral tissue. In embodiments, the device and/or methods described herein are used to monitor blood flow in a tissue of an organ of a mammal In a preferred embodiment, the tissue, or organ itself, is a tissue about to be transplanted or being transplanted or having been transplanted. The methods and devices described herein can be applied mutatis mutandis to measure fluid flow in a tissue, as opposed to blood flow. In an embodiment, the fluid is an organ viability-preserving fluid. In an embodiment of the organ is a lung, kidney, liver, pancreas, intestine, thymus or heart.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions encoded on a computer readable medium for execution by, or to control the operation of, data processing apparatus. The computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The methods, or portions thereof, processes and logic flows described in this specification can be performed by one or more programmable processors executing one or more computer programs to perform functions by operating on input data and generating output. The methods, or portions thereof, processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube), or LED (light emitting diode), or LCD/LED, or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

In embodiments, the methods as described herein when referring to cerebral blood flow can each be applied as stated in concert with, or simultaneously/contemporaneously with, a brain activity imaging/quantification method such as PET and fMRI methods, SPECT and CT. In embodiments the methods further comprise administering to the subject one or more agents, e.g. radionuclides, necessary to perform the brain activity imaging/quantification. In an embodiment, any two or more of the brain activity imaging/quantification methods can be used together to provide the detail on which the pattern of brain activity is identified. PET images demonstrate the metabolic activity chemistry of brain. A radiopharmaceutical, such as fluorodeoxyglucose, which includes both sugar and a radionuclide, is injected into the subject, and its emissions are measured by a PET scanner. The PET system detects pairs of gamma rays emitted indirectly by the positron-emitting radionuclide (tracer), which is introduced into the body on a biologically active molecule. Radiopharmaceuticals such as fluorodeoxyglucose as the concentrations imaged can be used as indication of the metabolic activity at that point. Magnetic resonance imaging (MRI) makes use of the property of nuclear magnetic resonance (NMR) to image nuclei of atoms inside the body, in this instance the brain. Strong magnetic field gradients cause nuclei at different locations to rotate at different speeds. 3-D spatial information can be obtained by providing gradients in each direction. In the embodiment of functional MRI (fMRI), the scan is used to measure the hemodynamic response related to neural activity in the brain.

In an embodiment the blood flow is measured in an arterial vessel. In another embodiment, the blood flow is measured in a venous vessel.

In a preferred embodiment of the methods and devices herein, the tissue in which the blood flow is measured is a tissue in a mammal In a most preferred embodiment the mammal is a human.

In one aspect of the invention, the flow sensor is as shown in FIG. 1A.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Results

### Introduction

A smart catheter has been developed which is capable of continuously monitoring multiple physiological and metabolic parameters (6-10). Here, the development of the smart catheter flow sensor (SCF) is described for real-time continuous measurement of cerebral perfusion. In a preferred embodiment, the SCF uses a 4-wire configuration to eliminate the effect of lead wires and operates in constant-temperature mode. The basic structure of an SCF is shown in FIG. 1(a). It comprises two components: a temperature sensor which is located outside the “thermal influence” area for temperature compensation during the heating period and a flow sensor which is heated, for example, to 2° C. above the medium temperature. FIG. 1(b) shows a preferred operational procedure: (1) the sensor is fully cooled down (region I); (2) medium temperature is measured by applying a small current without self-heating (region II); (3) during the initial heating period, the peak outputs are sampled for the subsequent medium thermal conductivity compensation (region III); (4) the sensor is heated to 2° C. above the baseline temperature and the outputs are compensated for the baseline temperature shifts with integrated temperature sensor (region IV); and (5) flow rate is derived with thermal conductivity compensation (region V).

The approach presented here has several advantages: (1) the SCF employs a periodic heating and cooling technique as opposed to the continuous heating. It recalibrates every period, so the outputs ensure zero drift; (2) it compensates baseline temperature shifts during the heating period with integrated temperature sensor and also medium thermal conductivity changes, so it provides more accurate outputs; and (3) compared to the current thermal diffusion flowmetry which can only monitor single parameter, the SCF is integrated with multiple microsensors to achieve the goal of multimodal monitoring.

### Materials and Methods

The flow sensor microelectrodes were fabricated by depositing a Au (1200 Å) layer with an adhesion layer of Ti (150 Å) on the 7.5 μm thick Kapton® film, followed by standard thin film lithography and etching processes. After that, the electrical leads of the flow sensors were electroplated with 2 μm thick Cu to reduce the lead resistances. Finally, 5 μm thick Parylene® film was deposited. The film with microsensors were cut into size and spirally rolled over the metal rode based on our previous work to form an intraventricular catheter (9). A developed flow sensor and signal conditioning interface circuit are shown in FIG. 2.

### Results

When the temperature of an electrically heated thermoresistive sensor increases, it loses thermal power to its surrounding until it reaches a thermal equilibrium in the presence of a moving fluid, which is defined by the following dynamic thermal energy balance equation:

$$\frac{{V}_{s}^{2}}{{R}_{s}} - ( a + b {υ}^{n} ) S ( {T}_{s} - {T}_{f} ) - m c \frac{d {T}_{s}}{d t} = 0 \qquad ( 1 )$$

where Vs is the voltage across the sensor, Rs is its resistance, Tₛ is the sensor temperature, T_{f} is the fluid temperature, m is the sensor mass, c is the specific heat, t is the time, b is the fluid velocity and a, b and n are constants.

For thin film Au, there is a linear dependence between the sensor's active element resistance and its temperature:

Rₛ=R₀(1+α(Tₛ−T₀)) (2)

where α is the temperature coefficient of resistance (TCR) of developed Au film.

Thus equation (1) can be rewritten as:

$$\frac{{V}_{s}^{2}}{{R}_{s}} - ( a + b {υ}^{n} ) S ( {T}_{s} - {T}_{f} ) - \frac{m c}{α {R}_{0}} \frac{d {R}_{s}}{d t} = 0 \qquad ( 3 )$$

As can be seen in equation (3), the time to reach thermodynamic equilibrium depends on several parameters, such as SCF resistance, surface area, TCR and its thermal capacity. The SCF resistance is 207±5.6 Ω, surface area is 1.8 mm2 and TCR is 3200 ppm/K.

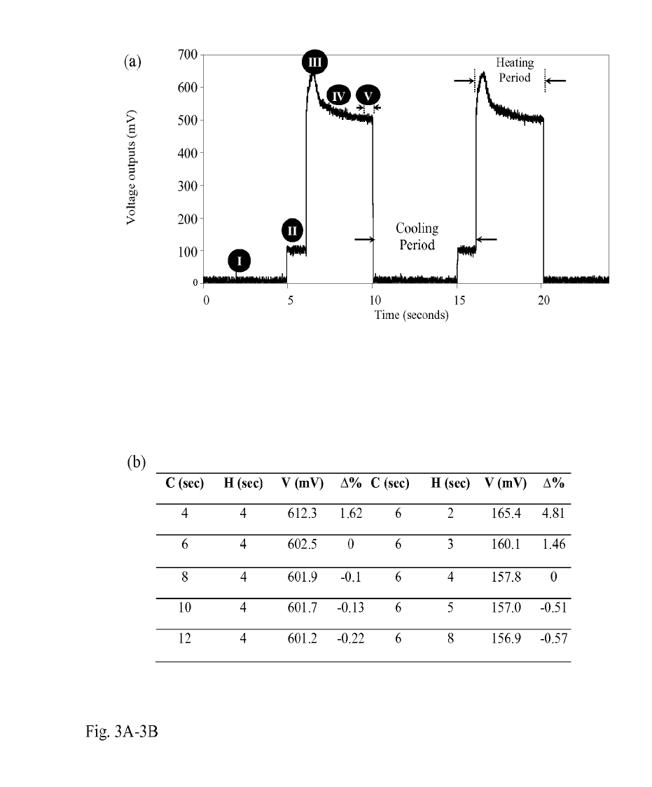

Thermodynamic equilibrium tests were performed to determine the time required to reach an equilibrium state, where the SCF was cooled down, the target resistance was recalculated and the constant temperature mode was restarted. FIG. 3(a) shows the timing diagram of the SCF outputs. Different times were applied for cooling and heating peirods and the SCF outputs were recorded in FIG. 3(b). To optimize for both high temporal resolution and accurate outputs, the cooling and the heating periods are set to 6 and 4 seconds, respectively. Even if the operation periods are doubled, the produced errors for SCF outputs are less than 0.22% and 0.57% in the cooling and heating period, respectively.

One of the most important sources of error in measuring flow rate is the change in the sensor calibration due to changes in the medium temperature. Periodic heating method requires only a single temperature point, where baseline conditions are established before the piont of heating. So, the measurement is unaffected by spatial temperature gradients in comparison with continuous heating method (11). However, the flow measurements can still be disturbed if temperature varation occurs during the heating period. The temperature in physiologic systems is not constant, with excursions of up to 0.5-1° C. over periods of minutes. Such variations can potentially disturb the flow measurements, especially if it happens during the heating peirod. This effect can be compensated for by integrating a separate temperature sensor (SCT) (12) outside the region of “thermal influence” of the heated SCF to continuously monitor baseline temperature.

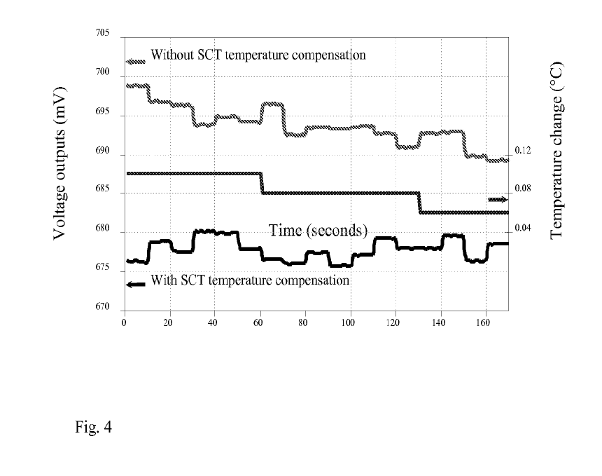

SCF outputs with and without temperature compensation during the heating period were measured by putting the SCF in the cooling flowing system. The results are shown in FIG. 4. The middle line indicates the medium temperature changes measured by the integrated SCT between the beginning and the end of the heating period (4 seconds) and the SCF ouputs update once per 10 seconds. As can be seen in FIG. 4, the outputs without temperature compensation are higher than the outputs with compensation. This is because the calculated target resistance at the beginning of heating period does not follow the medium temperature drop (0.025° C./sec for 0-60 seconds; 0.02° C./sec for 60-130 seconds; 0.015° C./sec for 130-170 seconds), so the sensor will be kept at higer temperature than its original value above the medium. It can be treated as if the effective target resistance has been increased. The uncompensated output is also linearly related to the measured temperature change because the temperature change is proportional to the effective target resistance. The produced error due to the medium temperature drop was bigger than 5 ml/100 gram-min. In contrast, the compensated output remains constant regardless of the ambient temperature change since the target resistance is updated during the heating period. The result confirms the efficacy of utilizing the temperature compensation method to obtain accurate outputs.

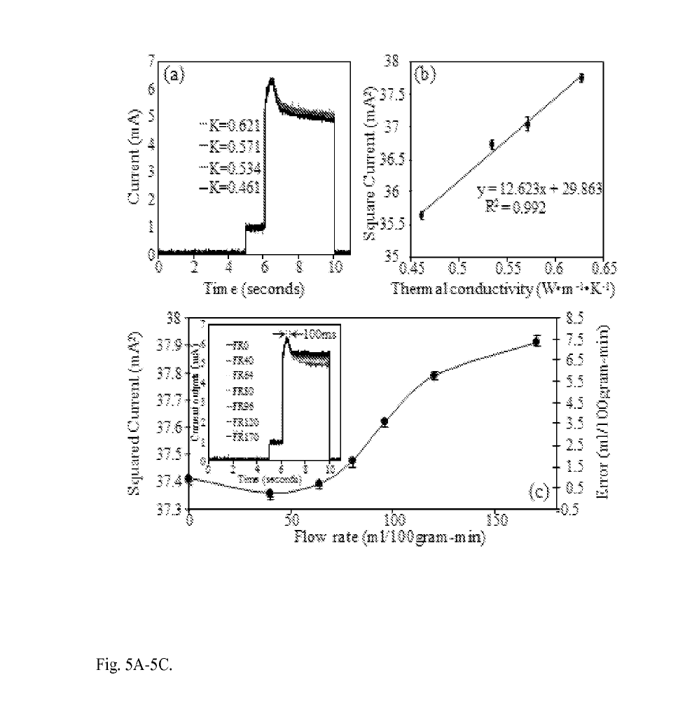

In perfused tissue, thermal transport is due to the contribution of two mechanisms: thermal conduction by the tissue property of thermal conductivity and convection due to microvascular fluid flow. Therefore, it is necessary to separate thermal conduction and convection components in order to achieve an adequate and reliable measurement of flow rate. Intrinsic thermal conductivity is a dynamic parameter which changes over time with the level of tissue hydration. Hence, frequent measurement and compensation of tissue thermal conductivity will greatly improve the SCF accuracy. In the approach disclosed herein, thermal conductivity compensation is achieved by sampling the startup current required to restablish the constant temperature operation. The current is sampled around its peak value (100 ms time window) where the sensor temperature is less than 0.15° C. above the target temperature. Two current samples are recorded and and their average squared number is used for thermal conductivity compensation. Transient current responses of the SCF in the medium with different thermal conductivity (glucose solutions with thermal conductivity of 0.621, 0.571, 0.534 and 0.461 W·m-1·K-1) were measured at the beginning of heating period (FIG. 5(a)). The squared transient current required to maintain the flow sensor at a constant temperature elevation (2° C.) was linearly related to the medium thermal conductivity as shown in FIG. 5(b). In such a time window, the startup current is more dependent on the medium intrinsic thermal conductivity than the flow rate, as shown in the inset of FIG. 5(c). FIG. 5(c) shows the squared currents plotted against different flow rates. The associated errors in unit of flow rate are also illustrated. Squared currents are nearly independent of flow rates (<current system flow accuracy 5 ml/100 gram-min) under 100 ml/100 gram-min, enabling estimation of intrinsic thermal conductivity without no-flow calibration.

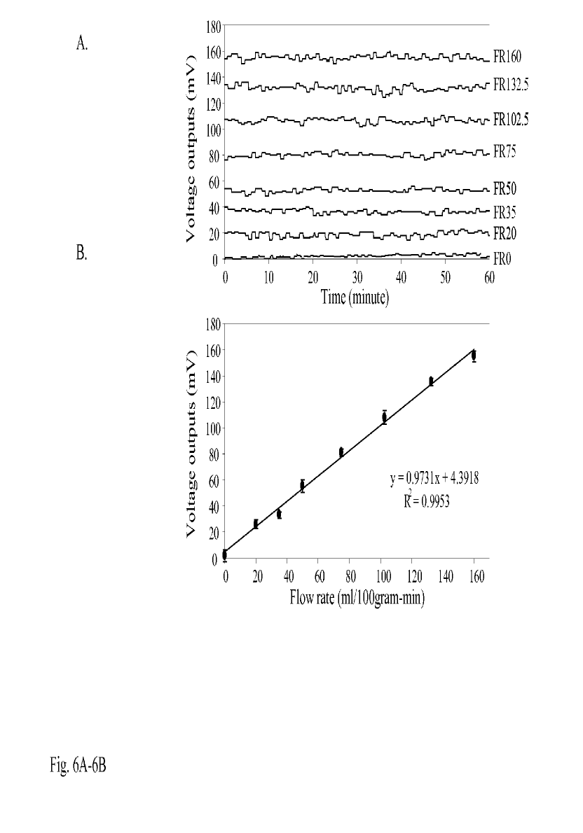

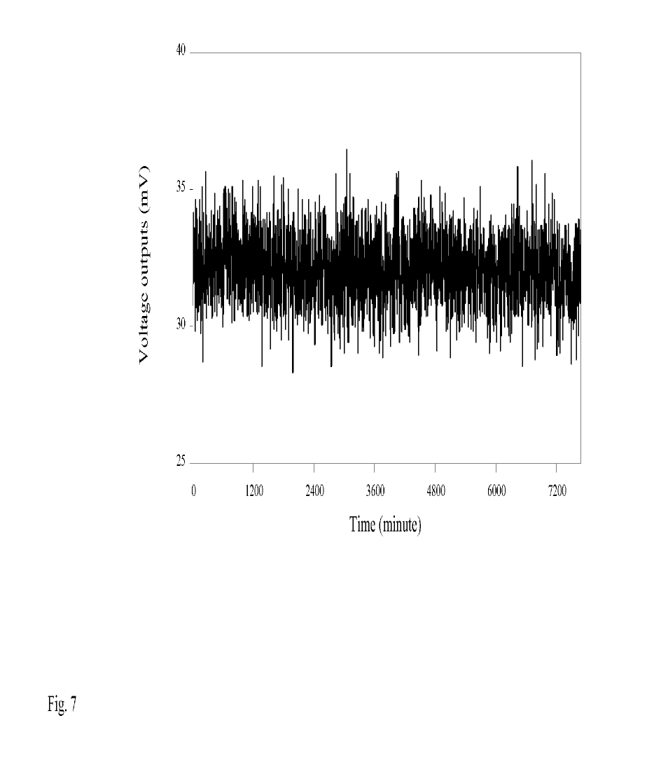

A tissue perfusion simulator similar to that of Thalayasingham and Dely (13) was constructed to measure the SCF outputs changed under different flow rates. Glucose water (k=0.534 W·m-1·K-1) was directed up through a tube, into a piece of sponge (10 ml) so that flow through the sponge was radially outwards. The flow rate was varied from 0.2 ml-20 ml (equivalent to 2 ml-200 ml/100 gram-min) and the SCF outputs were measured. The result is shown in FIG. 6. The sensitivity of SCF is 0.973 mV/ml/100 gram-min in the range from 0 to 180 ml/100 gram-min with the linear coefficient of R²=0.9953. The SCF can reach resolution of 0.25 ml/100 gram-min and accuracy better than 5 ml/100 gram-min of full scale. The long-term tests under the flow of 30 ml/100 gram-min for 5 days are shown in FIG. 7. The outputs are well within the system accuracy (5 ml/100 gram-min) without drift.

The sensor is advantageous over the art. For example:
(1) It calibrates itself (e.g. every 5-10 seconds) and approaches virtually a zero drift for long-term continuous monitoring. It provides very reliable data with MEMS-based thin film sensors whose tolerance is not as high as bulky material.
(2) It can use a 4-wire configuration to eliminate lead wire effect and employs ratiometric measurement to deduce the resistance of the sensor. Hence it is more precise as compared to the bridge-type thermal diffusion flow sensor.
(3) It compensates for baseline temperature shifts during the heating period with an integrated temperature sensor; and also for medium thermal conductivity changes, so it provides more accurate measurements.
(4) The principle of thermal diffusion flowmetry requires that there is perfect thermal contact between the sensor and tissue. However, brain tissue moves and there is a high possibility that sometimes the sensor and tissue are separated. The approach herein recalibrates every few seconds, and documents thermal conductivity which can differentiate whether the sensor is in good contact with the tissue.
(5) Compared to the current thermal diffusion flowmetry which can only monitor single parameter, the new sensor can be integrated with multiple microsensors to achieve the goal of multimodality monitoring.

In conclusion, the periodic heating mechanism of a 4-wire configuration flow sensor gives reliable results and ensures zero drift for long-term continuous monitoring. Using a series of data compensation algorithms, convection and conduction components are acquired separately; and temporal baseline temperature shifts during the heating period are compensated. Furthermore, the ability of the flow sensor to be integrated with multiple microsensors into a single catheter makes it an attractive choice for multimodality monitoring.

### REFERENCES

1. D. Mette, R. Strunk, M. Zuccarello, Translational stroke research 2, 152 (2011).
2. S. C. Lee, J. F. Chen, S. T. Lee, J Clin Neurosci 12, 520 (2005).
3. A. Dagal, A. M. Lam, Curr Opin Anesthesio 24, 131 (2011).
4. G. Rosenthal, R. O. Sanchez-Mejia, N. Phan, J. C. Hemphill, C. Martin, G. T. Manley, J Neurosurg 114, 62 (2011).
5. F. Verdú-López, J. M. González-Darder, P. González-López, and L. Botella Macia, Neurocirugia 21, 373 (2010).
6. C. Li, J. Han, C. H. Ahn, Biosens Bioelectron 22, 1988 (2007).
7. C. Li, P. M. Wu, J. Han, C. H. Ahn, Biomed Microdevices 10, 671 (2008).
8. C. Li, P. M. Wu, W. Jung, C. H. Ahn, L. A. Shutter, R. K. Narayan, Lab Chip 9, 1988 (2009).
9. C. Li, L. A. Shutter, P. M. Wu, C. H. Ahn, R. K. Narayan, Lab Chip 10, 1476 (2010).
10. C. Li, P. M. Wu, L. A. Shutter, R. K. Narayan, Appl Phys Lett 96, 053502-1 (2010).
11. C. Li, P. M. Wu, Z. Wu, C. H. Ahn, J. A. Hartings, R. K. Narayan, Proc. Of the 10th IEEE Sensors Conference (2011), Accepted.
12. C. Li, P. M. Wu, Z. Wu, C. H. Ahn, D. LeDoux, L. A. Shutter, J. A. Hartings, R. K. Narayan, Biomed Microdevices DOI: 10.1007/s10544-011-9589-4 (2011).
13. S. Thalayasingham, D. T. Delpy, Med. Biol. Comput. 27, 394 (1989).

1-56. (canceled)

57. A sensor for monitoring blood flow in a biological tissue, the sensor comprising (a) a flow sensor comprising a first microelectrode, and (b) a temperature sensor comprising a second microelectrode, wherein (a) and (b) are arranged on a flexible substrate and wherein (a) is positioned relative to (b) such that when (a) is heated to a stable target temperature of 0.5° C. to 3° C. above the temperature of the tissue in which the sensor is situated, (b) is not within the temperature field generated by (a).

58. The sensor for monitoring blood flow of claim 57, wherein the flow sensor comprising a first microelectrode comprises a 4-wire configuration.

59. The sensor for monitoring blood flow of claim 57, wherein the flow sensor is a constant-temperature flow sensor.

60. The sensor for monitoring blood flow of claim 57, wherein the temperature sensor quantitates the temperature of the medium in which the sensor for monitoring blood flow is situated, and wherein the sensor for monitoring blood flow is a component of an electrical circuit such that the output of the flow sensor is corrected for changes in temperature of the medium by the output of the temperature sensor.

61. The sensor for monitoring blood flow of claim 57, wherein the electrical circuit comprises an interface circuit.

62. The sensor for monitoring blood flow of claim 57, wherein the medium comprises the biological tissue.

63. The sensor for monitoring blood flow of claim 57, wherein the medium comprises blood in the biological tissue.

64. The sensor for monitoring blood flow of claim 57, wherein the microelectrodes are each continuous with an electrically-conducting wire, each of which wires are electroplated with a material to reduce lead resistances thereof

65. The sensor for monitoring blood flow of claim 64, wherein the electrically-conducting wires are electroplated with copper.

66. The sensor for monitoring blood flow of claim 57, wherein the microelectrodes comprise gold.

67. The sensor for monitoring blood flow of claim 57, wherein the microelectrodes comprise titanium.

68. The sensor for monitoring blood flow of claim 65, wherein the microelectrodes and/or electrically-conducting wires further comprise a flexible insulator coating.

69. The sensor for monitoring blood flow of claim 68, wherein the flexible insulator coating comprises poly(4,4′-oxydiphenylene-pyromellitimide).

70. The sensor for monitoring blood flow of claim 57, which flow sensor is capable of being heated by conduction of electric current through the microelectrode

71. The sensor for monitoring blood flow of claim 57, wherein (a) is capable of being heated to 0.1° C. to 3.5° C. above the temperature of the tissue in which the sensor is situated for a continuous time period of up to 20 seconds.

72. The sensor for monitoring blood flow of claim 57, wherein the flexible substrate comprises one or more polymers selected from the group consisting of polyimide, poly(p-xylylene), and polyvinylidene fluoride trifluoroethylene (PDVF-TrFE).

73. The sensor for monitoring blood flow of claim 57, wherein the sensor is integrated on an assembly further comprising one or more of a pressure sensor, a pH sensor, a glucose sensor, a microdialysis probe, an oxygen sensor, a lactate sensor, a pyruvate sensor, a glutamate sensor, and a carbon dioxide sensor.

74. The sensor for monitoring blood flow of claim 57, wherein the sensor is fabricated as a flexible spirally-rolled polymer microtube.

75. A method for determining a blood flow in a tissue of subject comprising measuring the blood flow using the sensor for monitoring blood flow of claim 57 situated in the tissue of the subject.

76. A system for monitoring a blood flow in a subject, comprising:
one or more data processing apparatus coupled to a sensor for monitoring blood flow of claim 57; and
a computer-readable medium coupled to the one or more data processing apparatus having instructions stored thereon which, when executed by the one or more data processing apparatus, cause the one or more data processing apparatus to perform a method of claim 75 so as to monitor the blood flow in a subject.
